(19) United States
(12) Patent Application Publication
(10) Pub. No.: US 2015/0282980 A1
(43) Pub. Date: Oct. 8, 2015

(54) VULVA COOLING RELIEF PAD

(71) Applicants: Bamidele Ogunleye, Bloomington, IL (US); Terry Ogunleye, Bloomington, IL (US)

(72) Inventors: Bamidele Ogunleye, Bloomington, IL (US); Terry Ogunleye, Bloomington, IL (US)

(21) Appl. No.: 14/247,805

(22) Filed: Apr. 8, 2014

(51) Int. Cl. A61F 7/10

(52) U.S. Cl. CPC A61F 7/10; A61F 7/106; A61F 2007/108; A61F 2007/005

(57) ABSTRACT

A vulva cooling pad to relieve discomfort in a female vulva region comprising a body, where the body includes a top surface and a bottom surface, where the top surface is domed shaped to fit against the vulva region; an inner cavity of the body; and at least one reservoir within the inner cavity, where the reservoir houses a cooling substance. The vulva cooling pad is filled with a cooling substance that is either frozen or instantly cooled to relieve pain, itchiness, swelling, burning and irritation in the vulva region.

### BACKGROUND OF THE INVENTION

1. Field of the Invention

The present invention relates to a sanitary pad that to relieve the pain and discomfort experienced after trauma to the vulva area.

2. Description of Related Art

The birth of a baby is one of the most joyous times in a woman's life. The new baby brings happiness to everyone around her, along with a great deal of responsibility. While the birth is a blessing, recovery is also a considerable venture for the mother. The babies are born either vaginally or by cesarean section. The cesarean section is very invasive, and requires a substantial recovery period. A vaginal delivery, while it does have a considerably shorter recovery period, the initial discomfort is significant. After a vaginal birth the mother's vulvar region is very sore, swollen, and tender. While the body is made to deliver the babies, the immediate trauma to the area is serious and requires a bit of TLC.

To help with the pain and discomfort of a vaginal birth, nurses and doctors typically give the mother an ice pack or cooling pack to place in their underwear. While these packs provide temporary relief they are often uncomfortable themselves unable to properly fit to the woman's body. Also, the cooling relief is short because the ice melts so quickly. Alternatively, some women find relief is making their own cooling pads. They soak a maxi pad in witch hazel, and then freeze it. The frozen witch hazel is both cooling and pain relieving. Again, this relief is short lived because the witch hazel melts quickly, and ends up creating a bigger mess with the melted liquid.

Therefore it would be beneficial in the art to provide a post-delivery pain relieving device that lasts for an extended period of time before having to discard or refreeze. It would also be desirable in the art to provide a pain relieving device that fits or conforms to the wearer's body for a comfortable fit.

### SUMMARY OF THE INVENTION

In view of the foregoing disadvantages inherent in the prior art, the general purpose of the present invention is to provide a vulva cooling pad to relieve discomfort in a female vulva region, configured to include all of the advantages of the prior art, and to overcome the drawbacks inherent therein.

Accordingly, an object of the present invention is to a vulva cooling relief pad comprising a domed body that fits against the vulva within the underwear of a wearer.

Another object of the present invention is to provide a vulva cooling relief pad with an internal reservoir that houses a cooling substance, like a refrigerant gel or an instant cooling chemical, to provide extended cooling for the wearer.

To achieve the above objects, in an aspect of the present invention, a vulva cooling pad is described comprising a body, where the body includes a top surface and a bottom surface, where the top surface is domed shaped to fit against the vulva region; an inner cavity of the body; and at least one reservoir within the inner cavity, where the reservoir houses a cooling substance. The vulva cooling pad is filled with a cooling substance that is either frozen or instantly cooled to relieve pain, itchiness, swelling, burning and irritation in the vulva region.

These together with other aspects of the present invention, along with the various features of novelty that characterize the present invention, are pointed out with particularity in the claims annexed hereto and form a part of this present invention. For a better understanding of the present invention, its operating advantages, and the specific objects attained by its uses, reference should be made to the accompanying drawings and descriptive matter in which there are illustrated exemplary embodiments of the present invention.

### BRIEF DESCRIPTION OF THE DRAWINGS

The advantages and features of the present invention will become better understood with reference to the following detailed description and claims taken in conjunction with the accompanying drawings, wherein like elements are identified with like symbols, and in which:

FIG. 1 depicts a side view of a vulva cooling relief pad in accordance with an exemplary embodiment of the present invention;

FIG. 2 depicts a top view of a vulva cooling relief pad in accordance with an exemplary embodiment of the present invention; and

FIG. 3 depicts a bottom view of a vulva cooling relief pad in accordance with an exemplary embodiment of the present invention.

Like reference numerals refer to like parts throughout the description of several views of the drawings.

### DETAILED DESCRIPTION OF THE DRAWINGS

The present invention relates to a sanitary pad that to relieve the pain and discomfort experienced after trauma to the vulva area. The present invention provides a vulva cooling relief pad to provide post-surgical relief for the female genital region. The vulva cooling relief pad is an oval or football shaped device with internal cooling properties. The pad is positioned against the wearer's body like a maxi pad, and is curved and raised in the middle. A cooling gel is incorporated into the pad to reduce inflammatory conditions and facilitate extensive periods of use by promoting optimal comfort. The vulva cooling relief pad may be reusable or disposable depending on the preferred materials used.

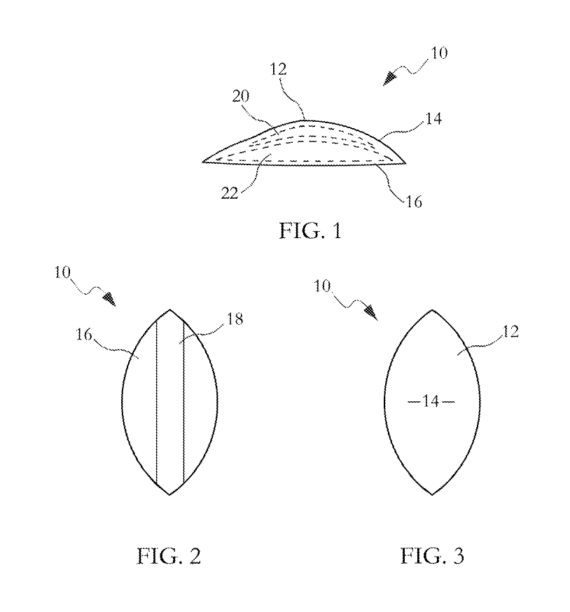

Turning now descriptively to the drawings, referring to FIG. 1, a side view of a vulva cooling relief pad 10 is shown in accordance with an exemplary embodiment of the present invention. The vulva cooling relief pad 10 includes a body 12 with a domed top surface 14 and a flat bottom surface 16. The body 12 may comprise a generally oval shape or a football shape with tapered ends, as more clearly illustrated in FIGS. 2 and 3. The body 12 includes a smooth exterior texture for optimal comfort against a wearer's body. The domed top surface 14 conforms to the shape of the user's vulva area. The body 12 may be 8-14 centimeters long, 3-6 centimeters wide and 1-4 centimeters tall. Preferably, the body 12 measures 10 cm long, 4 cm wide, and 2 cm tall.

The body 12 includes an inner cavity that may house one or more reservoirs, a first reservoir 20 and a second reservoir 22. The first 20 and second reservoirs 22 may be filled with one or more cooling substances. The cooling substances may be water/ice, refrigerant gel, instant cooling chemicals like ammonium nitrate or calcium ammonium, and the like. The cooling substances enable the body 12 to remain cool for an extended period of time, even when exposed to the warmth of the user's body. The vulva cooling relief pad 10 may be disposable, especially for use with instant cooling chemicals. This way the user may activate the cooling substance, use the pad and then throw it away. Alternative, the pad 10 may be reusable where the top surface 14 and the bottom surface 16 are made from a waterproof material. This way the pad 10 may be washed, and refrozen before the next use.

Referring now to FIG. 2, a top view of the vulva cooling relief pad 10 is shown in accordance with an exemplary embodiment of the present invention. The body 12 is shaped as an oval for an anatomical fit. The top surface 14 is smooth, without sharp edges or abrasive surfaces, so that the skin of the wearer is not additionally injured or hurt during use. Referring to FIG. 3, a bottom view of the vulva cooling relief pad 10 is shown in accordance with an exemplary embodiment of the present invention. The bottom surface 16 is flat to rest within the user's underwear or disposable undergarment. An adhesive strip 18 is centrally positioned vertically along the bottom surface 16. The adhesive strip 18 may secure the body 12 against the undergarments to ensure that the vulva cooling relieve pad 10 does not shift or fall out while wearing.

The vulva cooling relief pad provides comfort from pain, swelling, burning and itching of the vulvar or vaginal region due to its ability to sustain a cool temperature for long periods of time. Additionally, the pad conforms and maintains its shape to securely and comfortably rest against the vulva. The vulva cooling relief pad is especially useful for women after vaginal birth deliveries, and those who suffer from vulvodynia. The cooling properties of the internal cavity cool, numb and relieve the pain commonly associated with these conditions.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. A vulva cooling pad to relieve discomfort in a female vulva region comprising:
a. a body, where the body includes a top surface and a bottom surface, where the top surface is domed shaped to fit against the vulva region;
b. an inner cavity of the body; and
c. at least one reservoir within the inner cavity, where the reservoir houses a cooling substance.

2. The vulva cooling pad according to claim 1, where the inner cavity includes two reservoirs.

3. The vulva cooling pad according to claim 1, where the reservoir contains a refrigerant gel.

4. The vulva cooling pad according to claim 1, where the reservoir contains an instant cooling chemical.

5. The vulva cooling pad according to claim 1, where the bottom surface includes an adhesive strip to secure the body to an undergarment.

6. The vulva cooling pad according to claim 1, where the body is generally football shaped.

7. The vulva cooling pad according to claim 1, where the body is 10 cm long, 4 cm wide and 2 cm tall.

8. The vulva cooling pad according to claim 1, where the body includes a waterproof outer surface.

9. The vulva cooling pad according to claim 8, where the waterproof outer surface enables the body to be washable and therefore reusable.

10. The vulva cooling pad according to claim 1, where the body is disposable.
